(19) United States
(12) Patent Application Publication
(10) Pub. No.: US 2006/0122260 A1
(43) Pub. Date: Jun. 8, 2006

(54) Intermediates for aromatic polyketides as selective anticancer and antiviral agents

(76) Inventors: Tero Kunnari, Forby (FI); Matti Vuento, Helsinki (FI)

Correspondence Address: BIRCH STEWART KOLASCH & BIRCH, PO BOX 747, FALLS CHURCH, VA, 22040-0747, US

(21) Appl. No.: 10/533,378

(22) PCT Filed: Nov. 19, 2003
(86) PCT No.: PCT/FI03/00885

§ 371 Date: Apr. 29, 2005

(30) Foreign Application Priority Data
Nov. 20, 2002 (FI) 20022074

(51) Int. Cl. A61K 31/353; C07D 493/02

(52) U.S. Cl. 514/453; 549/384

(57) ABSTRACT

The present invention relates to the finding of potentiality of aromatic polyketide intermediates in drug development and, in specific, use of these compounds in development of antiviral or anticancer medicines.

### FIELD OF THE INVENTION

The present invention relates to the finding of potentiality of aromatic polyketide intermediates in drug development and, in specific, use of these compounds in development of antiviral or anticancer medicines.

### BACKGROUND OF THE INVENTION

The term “aromatic polyketides” describes a large number of different chemical compounds originating from microbes. Common features are found in their biosynthetic pathways and in the genomics. These compounds are also called Type II polyketides. Typically, aromatic polyketides are bioactive and show a wide range of different action mechanisms. For this reason, toxicity properties are common for this class of compounds. See the review article of Thomas (2001) and the references therein for further information.

Aromatic polyketides have been used in the treatment of bacterial infections and cancer during more than half a century. The reason why only these two therapeutic areas have been reached is due to the screening against these diseases, while based on their bioactivity profile these compounds exhibit potentiality to other therapeutic areas as well. However, toxic properties hamper their use in medical care. Classical examples of aromatic polyketides in clinical use are doxorubicin used for cancer treatment and tetracycline, which is in use for bacterial infections. See references like Gewirtz (1999) and Chopra et al. (1992).

Aromatic polyketides are fused-ring compounds, which are formed by the action of polyketide synthases (PKSs), which catalyse the assembly of small carbon chain units and folding of poly-beta-ketoacyl intermediates. Several different possibilities in the polyketide pathway and, in addition, post-polyketide modifications are the basis for the huge diversity of the class. A very typical structural feature is a quinonic substructure, which might also be one reason for the several toxic properties of these compounds. The quinonic structure is partially generated by additional oxygenation reactions, in addition to the carbonyl group of the polyketide assembly. Early intermediates of aromatic polyketides are more or less unstable, and therefore there are not many bioactive studies with these compounds. However, genetic engineering of bacteria producing aromatic polyketides has enabled the isolation of intermediates, giving also a deeper understanding on the biosynthetic steps involved. See the references for biosynthetic intermediates (Thomas, 2001 and the references therein).

Streptomyces nogalater produces an aromatic polyketide, nogalamycin, which belongs to the group of anthracyclines. Several anthracyclines, represented by doxorubicin, are in clinical use for cancer treatment. Biosynthetic genes for the polyketide pathway of nogalamycin (sno) were disclosed in U.S. Pat. No. 5,986,077. When the genes were introduced into Streptomyces lividans, a typical laboratory strain producing endogenously an aromatic polyketide antibiotic, actinorhodin, and a spore pigment, both biosynthetic intermediates and hybrid products caused by concomitant action of sno genes together with S. lividans genes were detected. Two compounds, designated as S2502 and S2507, were hybrid products, even though their chemical nature was unique (Kunnari et al., 1999). The chemical structure of these products suggested that the compounds are not bioactive However, screening of the compounds against human viruses and against some cancer cell lines surprisingly revealed a valuable bioactivity profile for the compounds.

Compounds having chemical nature close to that of S2502 and S2507 are given in Table 1.

TABLE 1
S2502 and S2507 and similar compounds with a naphtoquinonic ring
system (structure search via STN, CAS registry database).

| Registry Number | Name/code | Bioactivity | Reference |
|---|---|---|---|
| 247933-25-1 | S2502 | Not detn | Kunnari et al. 1999 |
| 247933-24-0 | S2507 | Not detn | Kunnari et al. 1999 |
| 99955-87-0 | — | Not described | Hanumaiah et al. 1985 |
| 99933-37-6 | — | Not described | Hanumaiah et al. 1985 |
| 99933-36-5 | — | Not described | Hanumaiah et al. 1985 |

The compounds discovered by Hanumaiah et al. (1985) differ from S2502 and S2507 in several positions. Major differences of the body are a quinonic structure in the first aromatic ring, keto-groups at positions C8 and C11 and, in addition, a keto-group at C17 (see the structure diagram with numbering below).

The biosynthetic genes derived from Streptomyces nogalater, snoal-3,rA,aB,aC,aD,aE,gF as designated currently (Ylihonko et al., 1996), were introduced into S. lividans TK24. The compounds recovered were hybrid compounds showing the formulae:

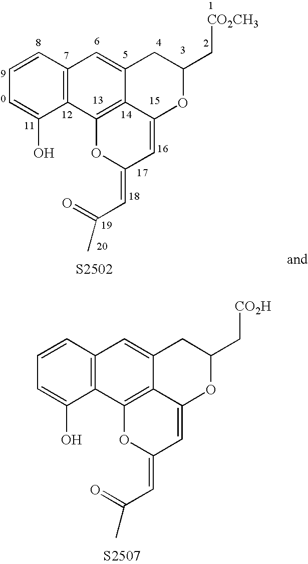

The —O— bridge (between carbons at positions 3 and 15) was suggested to be derived from actinorhodin biosynthesis (actVI genes) as was published by Kunnari et al. (1999). The compounds are distinctly products of a truncated pathway, and the absence of the quinonic structure suggested that the compounds are inactive. Typical cytotoxicity screen used (cell lines: MES-SA and MES-SA/Dx5) supported the postulation that the compounds fail to show anticancer activity. However, the compounds were later used in a virus model in order to study parvovirus endocytosis. Surprisingly, S2502 and S2507 showed a remarkable antiviral activity against parvovirus without disturbing the uninfected cells. This finding encouraged further studies on bioactivity.

### SUMMARY OF THE INVENTION

The present invention is thus directed to the aromatic polyketide compounds S2502 and S2507 and structural analogues or pharmaceutically acceptable salts thereof for use as medicaments, especially in the method of treatment of cancer of virus infection. These compounds can also be used as intermediates in the development of antiviral or anticancer medicines.

A further object of the invention is the use of the compounds S2502 and S2507 or their structural analogues or their pharmaceutically acceptable salts in the manufacture of a medicament for the treatment of cancer or virus infections.

A still further object of the invention is a pharmaceutical composition comprising a compound selected from the group consisting of S2502, S2507, structural analogues of these compounds and pharmaceutically acceptable salts of these compounds, in association with a pharmaceutically acceptable carrier. The invention also relates to a method for the treatment of cancer or virus infections, in which method to a patient in need of such treatment an efficacious amount of the above-identified pharmaceutical composition is administered.

### BRIEF DESCRIPTION OF THE DRAWINGS

FIG. 1 Infection percentages of S2507 (1), bafilomycin (2) and control (3) at different concentrations.

### DETAILED DESCRIPTION OF THE INVENTION

This invention relates to the finding of potentiality of two aromatic polyketide intermediates to be developed into antiviral or anticancer medicines. Of their chemical nature the compounds are not aromatic polyketides, even though they were generated by the genes involved in the biosynthesis of aromatic polyketides. Actually, the chemical class they include in is the naphtoquinone lactone class. The compounds S2502 and S2507 were isolated from the bacterial strain S. lividans TK24 carrying the plasmid pSY15. However, the low titre of the compounds hampered their purification process and a short-term strain improvement was carried out to give the strain S. lividans TK24/pSY15-B4 producing S2502 and S2507. The strain B4 is able to produce a few hundreds milligrams of the desired compounds per one litre of the cultivation medium. The compounds were isolated and purified for bioassays. Both of these related compounds show antiviral activity and selective anticancer activity.

While the chemical nature of the compounds did not indicate bioactivity, the unique chemical structures not identified in the studies on polyketides, encouraged us to use the compounds in assays available in the field. It is possible to screen the compounds against several different therapeutic areas, even though the chemical nature does not suggest bioactivity. S2507 was first used to study the effect on parvovirus, which has single-stranded DNA genome. The range of concentration used in the study was from 5 nM to 500 nM and, surprisingly, the effect was found with all of these dilutions. Interestingly, S2507 did not exhibit cytotoxic properties even with the highest concentration studied, 500 nM.

Structurally the compounds S2502 and S2507 are very similar, and the only difference is that S2507 is an acid, whereas S2502 is the methyl ester form of S2507. Both of the compounds are produced by fermentation of S. lividans TK24/pSY15-B4. S2502 is not soluble to water at pH 7 and S2507 is slightly soluble at the same pH. Both of the compounds are brownish yellow and stable in the typical conditions used in the purification process and in bioassays.

The results obtained from the parvovirus model encouraged us to carry out the bioactivity tests using human viruses as targets. Selected RNA- and DNA-viruses were tested; herpes simplex (HSV), adenovirus, influenza B, RS virus, cytomegalovirus (CMV), and CBV₅ virus. The virus tests indicated that both S2502 and S2507 were active against the rest of the viruses tested, but only S2502 was active against CMV. The concentrations tested were 1 μM and 100 μM. The cytotoxic effect was not found using 1 μM of S2502 or S2507. Instead, 100 μM of S2502 was toxic to the cells used for CBV₅ and HSV infection, and 100 μM of S2507 was toxic to the cells used for HSV infection.

NCI tested the said compounds in the cell panel of 60 different human cancer cell lines and according to these results, the compounds, S2502 and S2507 were active against some melanoma cell lines.

The compounds are now in progress to be developed into drug candidates.

### EXPERIMENTAL

The compositions of the nutrient media and solutions used in the experiments are substantially described in U.S. Pat. No. 5,986,077, which is incorporated herein by reference.

### Example 1

### Production and Purification of S2502 and S2507

Streptomyces lividans TK24/pSY15 (DSM 9436) (Ylihonko et al., 1996) was mutated using a chemical mutagen NTG (N-methyl-N′-nitro-N-nitrosoguanidine) (Sigma) to generate the strain TK24/pSY15-B4. For mutagenization S. lividans TK24/pSY15 was cultured in 50 ml of TSB-medium in 250 ml Erlenmeyer flask, containing a string in the bottom of the bottle to disperse mycelia during cultivation. Cultivation of the said strain was carried out for two days, at 30° C., 330 rpm in a shaker. 1 ml of the culture was further inoculated to the next bottle containing 50 ml of TSB and the cultivation was continued for one day (30° C., 330 rpm). This younger culture was adjusted to pH 8.5 with 2% NaOH and 800 μg/ml of NTG was added to act on the cells for 20 minutes at 37° C. The mutagenized culture of the strain was divided into two tubes and pelleted by centrifugation (300 rpm, 10 minutes). Combined pellets were used to inoculate 50 ml of TSB medium. After one day (30° C., 330 rpm), the titre of the cell suspension was determined by plating suitable dilutions, 1:10-1:100000 on ISP4-plates. Colonies of the mutagenized culture were compared to the wild type and those exhibiting increased titre as compared with that of TK24/pSY15 were selected to repeat the cultivations. The best strain obtained was B4 and it produces about 100 to 500 mg/litre of the said compounds in typical laboratory conditions.

TK24/pSY15-B4 is pre-cultivated in E1 medium for two days in a shake flask and inoculated to 10 l jar fermentor working at 25° C., aeration 10 l/min and agitation 280 rpm. This cultivation procedure maximises the production of S2507 in favour of S2502. After seven days the broth is acidified with HCl to pH=3.250 g of XAD-7 absorbent (Amberlite XAD-7, Rohm and Haas, Philadelphia, USA) is added to the broth and it is stirred for one hour. The cells and the absorbent are separated with cross flow filtration from the supernatant, which is discarded. The cells and the absorbent are subsequently extracted with three 1.5 l portions of methanol. The methanol extracts are combined and filtrated through a glass funnel. 2 l of water and 2 l of petroleum ether (bp. 80-110° C.) are added to methanol and pH is adjusted to 7.5. The organic phase is washed with 1 l of water. Water phases are combined and washed twice with 0.5 l of petroleum ether. The water phase is filtered and pH is adjusted to 4.0 and subsequently extracted with 2×1 l of toluene. The organic phase is filtered and evaporated in vacuum to dryness. The yellow residue is dissolved in 100 ml of methanol, pH is adjusted to 3.0, and 5 ml of water is added. The solution is concentrated to 50 ml effecting the precipitation of the S2507. The mixture is allowed to stand for 24 hours at 4° C., after which the yellow precipitate is collected and dried under vacuum. The purity of the residue is over 90%.

### Example 2

### Physical Properties of S2502 and S2507

S2507 is a yellow solid compound, having a melting point above 250° C. It is a weakly acidic compound having pKa comparable to acetic acid (ca. 4.6). It is soluble to methanol, ethyl acetate and chloroform, and sparingly soluble to water and toluene. It is stable and non-reactive under normal laboratory conditions.

S2502 is a yellow solid compound, having a melting point above 250° C. It is the methyl ester of the S2507 acid. It is soluble to methanol, ethyl acetate and chloroform and insoluble to water and hexane. It is stable and non-reactive under normal laboratory conditions.

Physical properties (especially solubility and non-reactivity) support the drug development of the lead compounds.

### Example 3

### Antiviral Activity

### 3.1 Biological Activity of S2507 and S2502 in Animal Virus Model

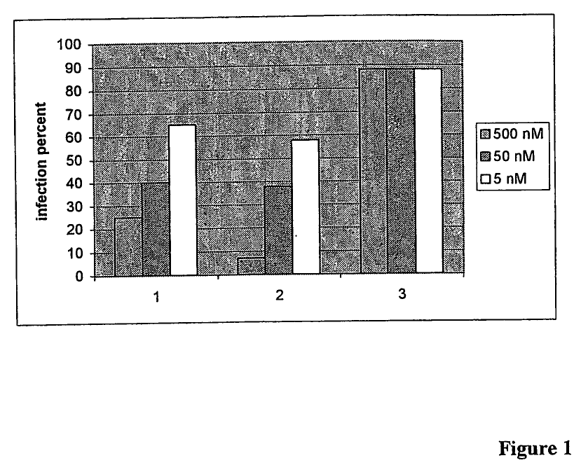

The compounds were initially tested against parvovirus. The results for S2507 and S2502 were alike. Both of the compounds were able to partially inhibit the infection of cells in the test system used. The chart in FIG. 1 presents the results of S2507 compared to negative control and positive control (bafilomycin). S2507 also indicated dose-activity correlation in the therapeutic concentration range of 500 nM to 5 nM.

### 3.1.1 Virus Production

Canine parvovirus type 2 (CPV-d) derived from a plasmid clone of virus was grown in NLFK cells in 175 cm² cell culture flasks (Nunc, Roskilde, Denmark) for 5 to 7 days, and then stored at −20° C. 300 ml of the virus culture medium was clarified by centrifugation and concentrated with ultrafiltration (30 kDa filter, Millipore Corporation, Bedford, Mass., U.S.A). CPV was pelleted with ultracentrifugation at 173 000 g for 1 h and resuspended in 0.9 ml of PBS, pH 7.4. The suspension was sonicated with low power and chloroform extracted.

### 3.1.2 Infectivity Screening

For infectivity screening studies cells grown on (diameter 13 mm) coverslips to 80% confluency were inoculated with CPV. The drug (1 μM or 500 nM, diluted to DMEM 2 coverslips/concentration) was added simultaneously with CPV and was present during the course of infection. Control samples were incubated without drug. 24 h post-infection (p.i.) the coverslips were dipped in phosphate-buffered saline (PBS), pH 7.4 and fixed with 4% paraformaldehyde in PBS. After permeabilization (1% BSA, 1% Triton X-100 and 0.01% sodium azide in PBS) for 15 min, the coverslips were incubated for 45 min at room temperature with primary antibody anti-CPV (Cornell #2, gift from Colin Parrish, Comell University, Ithaca, N.Y.) diluted in permeabilization buffer and rinsed several times. The coverslips were incubated with Alexa 488-conjugated anti-rabbit antibody (Molecular Probes, Eugene, Oreg., U.S.A) and texasred-X-conjugated wheat germ agglutinin (WGA) for 45 min. Before embedding with Mowiol with antifading agent DABCO (30 mg/ml, Sigma), the coverslips were rinsed several times. Samples were examined with a laser scanning fluorescence microscope (Zeiss, Axiovert 100M, LSM 510) using excitation/emission settings appropriate for the dye used. Infectivity was determined by counting the ratio of cells with fluorescent nuclei to total number of cells (stained with texasred-X-conjugated wheat germ agglutinin) (n=300) at 24 h p.i. by immunofluorescence with polyclonal antibody (Comell#2) to CPV.

### 3.2 Biological Activity of S2507 and S2502 in Human Virus Models

S2507 and S2502 were further tested in human virus models: herpes simplex (HSV), adenovirus, influenza B, RS virus, cytomegalovirus and CBV₅ virus. Compounds showed wide spectra of activity at test concentrations of 100 and 1 μM. The compounds were active when administered simultaneously or before an infection (prophylactic) and half an hour after the infection. The control cultivations without virus tolerated well the utilized drug concentrations. However, in HSV 100 μM concentration was toxic. Below 10% infection was observed at 1 μM in all viruses.

The results from the activity testing in human viruses are shown in Table 2.

TABLE 2
Results from the activity testing in human viruses

| | inhibition | inhibition | inhibition | inhibition |
|---|---|---|---|---|
| | % 1 μM | % 100 μM | % 1 μM | % 100 μM |
| Virus | (S2502) | (S2502) | (S2507) | (S2507) |
| Herpes (HSV) | 100 | toxic | 100 | toxic |
| Adeno | 90 | 100 | 90 | 100 |
| Influenza B | 100 | 100 | 100 | 100 |
| RSV | 100 | 100 | 100 | 100 |
| Cytomegalo | 100 | 100 | 0 | 0 |
| CBV₅ | 100 | toxic | 100 | 100 |

### Example 4

### Anticancer Activity

National Cancer Institute (NCI) tested both of the compounds in their 60-cell line panel (copy of their report available). NCI uses a 48-hour continuous drug exposure protocol. Cell viability and growth are estimated using a sulforhodamine B (SRB) protein assay. The screen is described on the web site: http://dtp.nci.nih.gov. Compounds showed very weak cytotoxicity well above the therapeutic concentrations in the virus panels in most cell lines. However, a mean value of ED₅₀ is at micro molar range against some melanoma cell lines (MAL-ME-3M, M14, SK-MEL-5). Surprisingly, the results obtained suggest a specific action mechanism (see Example 6).

### Example 5

### In Vivo Toxicity

A preliminary dose range finding study for toxicological effects using S2507 was carried out. A stock solution of S2507 was made in ethanol and diluted with water. The solubility of the compound is very poor and all dilutions were cloudy. The drug was injected once i.p. at doses 100, 50, 25, 12.5 and 6.25 mg/kg to two mice at each dose level. The mice were observed daily and weighed every 3 to 5 days for two weeks. The drug had no effect on the weight of the mice at these doses. Also, no outer signs of general illness were noticed.

### Example 6

### Studies on Action Mechanisms

The compounds are active against both RNA viruses and DNA viruses. The results suggest that antiviral activities of S2502 and S2507 reflect to the effect on endocytosis by these compounds.

In order to understand the mechanism of anticancer activity, the inhibition of topoisomerases by S2502 was studied. The topoisomerase assays were done using Topoisomerase I and II drug screening kits with human topoisomerase I and II (p170 form) enzymes, respectively, according to the manufacturer's (TopoGEN, Inc, Columbus, Ohio) instructions. The compound inhibited both topoisomerase I and II. Minimal inhibitory concentrations were 25 μM against Topoisomerase 1 and 50 μM against Topoisomerase II.

Topoisomerase inhibition is relevant to the cytotoxic concentration against special melanoma cell lines. However, the topoisomerase tests are not selective, suggesting that even though a compound is a topoisomerase inhibitor, there may be other more relevant mechanism(s) responsible for desired bioactivity profiles.

### REFERENCES

Chopra, I., Hawkey, P. M. and Hinton, M. 1992. Tetracyclines, molecular and clinical aspects. J. Antimicrob. Chemother. Mar; 29(3):245-277. Review.
Gewirtz, D. A. 1999. A critical evaluation of the mechanisms of action proposed for the antitumor effects of the anthracycline antibiotics adriamycin and daunorubicin. Biochem. Pharmacol. Apr 1; 57(7):727-741. Review
Hanumaiah, T., Rao, B. K., Rao, C. P., Rao, G. S. R, Rao, J. U. M., Rao, K. V. J., Marshall, D. S. and Thomson, R. H. 1985. Naphthalenes and naphtoquinones from Ventilago species. Phytochemistry. 24 (8): 1811-1815.
Kunnari, T., Kantola, J., Ylihonko, K., Klika, K., Mäntsälä, P. and Hakala, J. 1999. Hybrid compounds derived from combination of anthracycline and actinorhodin biosynthetic pathway. J. Chem. Soc. Perkin Trans. 2: 1649-1652.
Thomas, R. 2001. A biosynthetic classification of fungal and streptomycete fused-ring aromatic polyketides. Chembiochen., Sep 3; 2(9):612-627. Review.
Ylihonko, K, Tuikkanen, J., Jussila, S., Cong, L. and Mäntsälä, P. 1996. A gene cluster involved in nogalamycin biosynthesis from Streptomyces nogalater: sequence analysis and complementation of early blocked mutations of the anthracycline pathway. Mol. Gen. Genet. 251:113-120.

1. Compounds S2502 and S2507 having the formulae 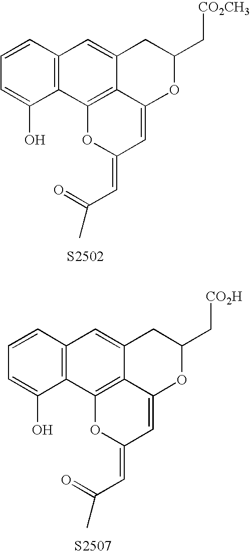
or their pharmaceutically acceptable salts for use as a medicament or in drug development.

2. The compounds S2502 and S2507 as defined in claim 1, or their structural analogues or their pharmaceutically acceptable salts for use in a method of prophylactic treatment of human or animal body.

3. Use of the compounds S2502 and S2507 as defined in claim 1, or their structural analogues or their pharmaceutically acceptable salts in the manufacture of a medicament for the treatment of virus infections.

4. Use of the compounds S2502 and S2507 as defined in claim 1, or their structural analogues or their pharmaceutically acceptable salts in the manufacture of a medicament for the treatment of cancer.

5. A pharmaceutical composition comprising a compound selected from the group consisting of the compounds S2502 and S2507 as defined in claim 1, structural analogues of these compounds and pharmaceutically acceptable salts of these compounds, in association with a pharmaceutically acceptable carrier.

6. A method for the treatment of cancer, comprising administering to a patient in need of such treatment an efficacious amount of the pharmaceutical composition according to claim 5.

7. A method for the treatment of virus infections, comprising administering to a patient in need of such treatment an efficacious amount of the pharmaceutical composition according to claim 5.
